(19) United States
(12) Patent Application Publication
(10) Pub. No.: US 2007/0276198 A1
(43) Pub. Date: Nov. 29, 2007

(54) Device,system,and method of wide dynamic range imaging

(76) Inventor: Horn Eli, Kiryat Motzkin (IL)

Correspondence Address: PEARL COHEN ZEDEK LATZER, LLP, 1500 BROADWAY 12TH FLOOR, NEW YORK, NY, 10036, US

(21) Appl. No.: 11/587,564

(22) PCT Filed:
(86) PCT No.: PCT/IL05/00441

Related U.S. Application Data
(60) Provisional application No. 60/564,938, filed on Apr. 26, 2004.

(51) Int. Cl. A61B 5/00

(52) U.S. Cl. 600/300

(57) ABSTRACT

A device, system and method for wide dynamic range imaging. An in-vivo imager may acquire first and second portions of an image, wherein the first and second portions are combinable into a wide dynamic range image. An in-vivo imaging device may determine local gain for a portion of an image acquired by an imager of the in-vivo imaging device.

### FIELD OF THE INVENTION

The present invention relates to the field of in-vivo sensing, for example, in-vivo imaging.

### BACKGROUND OF THE INVENTION

Devices, systems and methods for in-vivo sensing of passages or cavities within a body, and for sensing and gathering information (e.g., image information, pH information, temperature information, electrical impedance information, pressure information, etc.), are known in the art.

An in-vivo sensing system may include, for example, an in-vivo imaging device for obtaining images from inside a body cavity or lumen, such as the gastrointestinal (GI) tract. The in-vivo imaging device may include, for example, an imager associated with units such as, for example, an optical system, an illumination source, a controller, a power source, a transmitter, and an antenna. Other types of in-vivo devices exist, such as endoscopes which may not require a transmitter, and in-vivo devices performing functions other than imaging.

The in-vivo imaging device may transmit acquired image data to an external receiver/recorder, using a communication channel (e.g., Radio Frequency signals). The communication channel may limit the amount of data that may be transmitted per time unit from the in-vivo imaging device to the external receiver/recorder, e.g., due to bandwidth restrictions. Additionally, some images acquired in-vivo may suffer from color saturation.

### SUMMARY OF THE INVENTION

Various embodiments of the invention provide, for example, devices, systems and method to acquire in-vivo WDR images and/or to determine local gain, e.g., for a pixel or a portion of an image.

Some embodiments may include, for example, an in-vivo imaging device having an imager to acquire a WDR image, e.g., using double-exposures or multiple-exposures.

Some embodiments may include, for example, an imager to acquire first and second portions of a wide dynamic range image, wherein said first and second portions are combinable into said wide dynamic range image.

In some embodiments, for example, said first and second portions correspond to first and second aspects of said wide dynamic range image, respectively.

In some embodiments, for example, said imager is to acquire said first portion at a first light level and said second portion at a second light level.

In some embodiments, for example, said imager is to acquire said first portion at a first exposure time and said second portion at a second exposure time.

In some embodiments, for example, said imager is to acquire said first portion at a first gain and said second portion at a second gain.

In some embodiments, for example, said imager includes a plurality of groups of pixels including at least a group of low-responsivity pixels.

In some embodiments, for example, each of a set of color pixels includes at least one low-responsivity pixel.

In some embodiments, for example, said imager includes a first group of reduced-responsivity pixels to acquire said first portion, and a second group of pixels to acquire said second portion.

In some embodiments, for example, the number of pixels of the first group associated with a pre-defined color is equal to the number of pixels of the second group associated with said pre-defined color.

In some embodiments, for example, a pixel of said wide dynamic range image is represented using more than eight bits.

Some embodiments may include, for example, a processor to reconstruct said wide dynamic range image from said first and second portions.

Some embodiments may include, for example, a transmitter to transmit data of said first and second portions.

Some embodiments may include, for example, an imager having a plurality of groups of pixels including at least a group of low-responsivity pixels.

Some embodiments may include, for example, an in-vivo imaging device to determine local gain for a portion of an image acquired by an imager of said in-vivo imaging device.

In some embodiments, for example, said portion of an image includes a pixel.

In some embodiments, for example, said in-vivo imaging device is to determine gain of a first pixel based on gain of a second pixel.

In some embodiments, for example, said in-vivo imaging device is to determine local gain of a pixel based on a comparison of a value of said pixel with a threshold value.

In some embodiments, for example, said in-vivo imaging device is to create a representation of said local gain and at least a portion of a value of said pixel.

In some embodiments, for example, said representation is a floating-point type representation.

In some embodiments, for example, said in-vivo imaging device is to compress said representation.

In some embodiments, for example, the in-vivo device may include a transmitter to transmit the compressed representation.

In some embodiments, for example, said in-vivo imaging device is configured to avoid false saturation and/or an unstable data structure and/or over-quantization of data.

Some embodiments may include, for example, a receiver to receive from said in-vivo imaging device a representation of said local gain of a pixel and at least a portion of a value of said pixel.

Some embodiments may include, for example, a processor to reconstruct said value of said pixel and said gain of said pixel based on said representation.

Some embodiments may include, for example, acquiring in-vivo first and second portions of a wide dynamic range image, wherein said first and second portions are combinable into said wide dynamic range image.

Some embodiments may include, for example, acquiring said first portion at a first light level and said second portion at a second light level.

Some embodiments may include, for example, acquiring said first portion at a first exposure time and said second portion at a second exposure time.

Some embodiments may include, for example, acquiring said first portion at a first gain and said second portion at a second gain.

Some embodiments may include, for example, constructing said wide dynamic range image based on said first and second portions.

Some embodiments may include, for example, determining local gain for a portion of an in-vivo image.

Some embodiments may include, for example, determining local gain for a pixel of said in-vivo image.

Some embodiments may include, for example, determining gain of a first pixel based on gain of a second pixel.

Some embodiments may include, for example, determining local gain of a pixel based on a comparison of a value of said pixel with a threshold value.

Some embodiments may include, for example, creating a representation of local gain of a pixel and at least a portion of a value of said pixel.

Some embodiments may include, for example, creating a floating-point type representation of local gain of a pixel and at least a portion of a value of said pixel.

Some embodiments may include, for example, converting in-vivo a data item from a first bit-space to a second bit-space.

Some embodiments may include, for example, converting in-vivo said data item from said first bit-space to said second bit-space having a smaller number of bits.

Some embodiments may include, for example, creating a floating-point type representation of said data item.

Some embodiments may include, for example, creating a floating-point type representation of said data item, said floating-point representation having an exponent component corresponding to a gain value and a mantissa component corresponding to a pixel value.

Some embodiments may include, for example, creating in-vivo an oversized data item corresponding to in-vivo image data.

Some embodiments may include, for example, creating in-vivo said oversized data item having a first portion corresponding to a value of a pixel and a second component corresponding to local gain of said pixel.

Some embodiments may include, for example, converting in-vivo said oversized data item from a first bit-space to a second bit-space.

Some embodiments may include, for example, creating in-vivo a floating-point type representation of said oversized data item.

Some embodiments may include, for example, creating in-vivo a floating-point type representation of a data item acquired in-vivo.

Some embodiments may include, for example, creating said floating-point type representation having an exponent component corresponding to a gain value and a mantissa component corresponding to a pixel value.

Some embodiments may include, for example, discarding at least one least-significant bit of said pixel value.

Some embodiments may include, for example, compressing in-vivo said floating-point type representation.

Some embodiments may include, for example, an in-vivo imaging device which may be autonomous and/or may include a swallowable capsule.

Embodiments of the invention may allow various other benefits, and may be used in conjunction with various other applications.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings in which:

FIG. 1 is a schematic illustration of an in-vivo imaging system in accordance with some embodiments of the invention;

FIG. 2 is a schematic illustration of pixel grouping in accordance with some embodiments of the invention;

FIG. 3 is a schematic block diagram illustration of a circuit in accordance with some embodiments of the invention; and

FIG. 4 is a flow-chart diagram of a method of imaging in accordance with some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the invention will be described. For purposes of explanation, specific configurations and details are set forth in order to provide a thorough understanding of the invention. However, it will also be apparent to one skilled in the art that the invention may be practiced without the specific details presented herein. Furthermore, well-known features may be omitted or simplified in order not to obscure the invention.

It should be noted that although a portion of the discussion may relate to in-vivo imaging devices, systems, and methods, the present invention is not limited in this regard, and embodiments of the present invention may be used in conjunction with various other in-vivo sensing devices, systems, and methods. For example, some embodiments of the invention may be used, for example, in conjunction with in-vivo sensing of pH, in-vivo sensing of temperature, in-vivo sensing of pressure, in-vivo sensing of electrical impedance, in-vivo detection of a substance or a material, in-vivo detection of a medical condition or a pathology, in-vivo acquisition or analysis of data, and/or various other in-vivo sensing devices, systems, and methods. Some embodiments of the invention may be used not necessarily in the context of in-vivo imaging or in-vivo sensing.

Some embodiments of the present invention are directed to a typically swallowable in-vivo sensing device, e.g., a typically swallowable in-vivo imaging device. Devices according to embodiments of the present invention may be similar to embodiments described in U.S. patent application Ser. No. 09/800,470, entitled “Device And System For In-vivo Imaging”, filed on 8 Mar. 2001, published on Nov. 1, 2001 as U.S. Patent Application Publication Number 2001/0035902, and/or in U.S. Pat. No. 5,604,531 to Iddan et al., entitled “In Vivo Video Camera System”, each of which is assigned to the common assignee of the present invention and each of which is hereby fully incorporated by reference. Furthermore, a receiving and/or display system which may be suitable for use with embodiments of the present invention may also be similar to embodiments described in U.S. patent application Ser. No. 09/800,470 and/or in U.S. Pat. No. 5,604,531. Devices and systems as described herein may have other configurations and/or other sets of components. For example, the present invention may be practiced using an endoscope, needle, stent, catheter, etc.

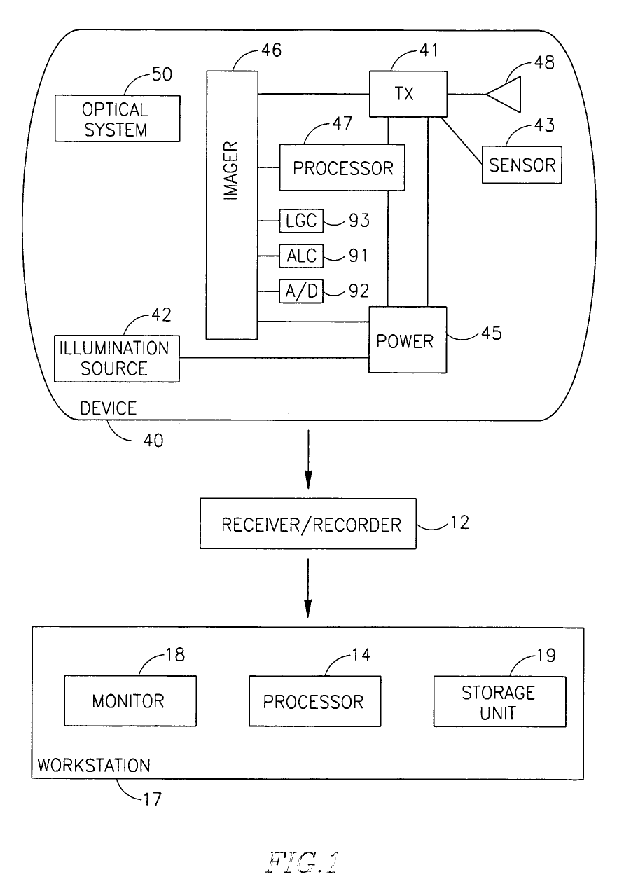

FIG. 1 shows a schematic illustration of an in-vivo imaging system in accordance with some embodiments of the present invention. In one embodiment, the system may include a device 40 having an imager 46, one or more illumination sources 42, a power source 45, and a transmitter 41. In some embodiments, device 40 may be implemented using a swallowable capsule, but other sorts of devices or suitable implementations may be used. Outside a patient's body may be, for example, an external receiver/recorder 12 (including, or operatively associated with, for example, an antenna or an antenna array), a storage unit 19, a processor 14, and a monitor 18. In one embodiment, for example, processor 14, storage unit 19 and/or monitor 18 may be implemented as a workstation 17, e.g., a computer or a computing platform.

Transmitter 41 may operate using radio waves; but in some embodiments, such as those where device 40 is or is included within an endoscope, transmitter 41 may transmit/receive data via, for example, wire, optical fiber and/or other suitable methods. Other known wireless methods of transmission may be used. Transmitter 41 may include, for example, a transmitter module or sub-unit and a receiver module or sub-unit, or an integrated transceiver or transmitter-receiver.

Device 40 typically may be or may include an autonomous swallowable capsule, but device 40 may have other shapes and need not be swallowable or autonomous. Embodiments of device 40 are typically autonomous, and are typically self-contained. For example, device 40 may be a capsule or other unit where all the components are substantially contained within a container or shell, and where device 40 does not require any wires or cables to, for example, receive power or transmit information. In one embodiment, device 40 may be autonomous and non-remote-controllable; in another embodiment, device 40 may be partially or entirely remote-controllable.

In some embodiments, device 40 may communicate with an external receiving and display system (e.g., workstation 17 or monitor 18) to provide display of data, control, or other functions. For example, power may be provided to device 40 using an internal battery, an internal power source, or a wireless system able to receive power. Other embodiments may have other configurations and capabilities. For example, components may be distributed over multiple sites or units, and control information or other information may be received from an external source.

In one embodiment, device 40 may include an in-vivo video camera, for example, imager 46, which may capture and transmit images of, for example, the GI tract while device 40 passes through the GI lumen. Other lumens and/or body cavities may be imaged and/or sensed by device 40. In some embodiments, imager 46 may include, for example, a Charge Coupled Device (CCD) camera or imager, a Complementary Metal Oxide Semiconductor (CMOS) camera or imager, a digital camera, a stills camera, a video camera, or other suitable imagers, cameras, or image acquisition components.

In one embodiment, imager 46 in device 40 may be operationally connected to transmitter 41. Transmitter 41 may transmit images to, for example, external transceiver 12 (e.g., through one or more antennas), which may send the data to processor 14 and/or to storage unit 19. Transmitter 41 may also include control capability, although control capability may be included in a separate component, e.g., processor 47. Transmitter 41 may include any suitable transmitter able to transmit image data, other sensed data, and/or other data (e.g., control data) to a receiving device. Transmitter 41 may also be capable of receiving signals/commands, for example from an external transceiver 12. For example, in one embodiment, transmitter 41 may include an ultra low power Radio Frequency (RF) high bandwidth transmitter, possibly provided in Chip Scale Package (CSP).

In some embodiment, transmitter 41 may transmit/receive via antenna 48. Transmitter 41 and/or another unit in device 40, e.g., a controller or processor 47, may include control capability, for example, one or more control modules, processing module, circuitry and/or functionality for controlling device 40, for controlling the operational mode or settings of device 40, and/or for performing control operations or processing operations within device 40. According to some embodiments, transmitter 41 may include a receiver which may receive signals (e.g., from outside the patient's body), for example, through antenna 48 or through a different antenna or receiving element. According to some embodiments, signals or data may be received by a separate receiving device in device 40.

Power source 45 may include one or more batteries or power cells. For example, power source 45 may include silver oxide batteries, lithium batteries, other suitable electrochemical cells having a high energy density, or the like. Other suitable power sources may be used. For example, power source 45 may receive power or energy from an external power source (e.g., an electromagnetic field generator), which may be used to transmit power or energy to in-vivo device 40.

Optionally, in one embodiment, transmitter 41 may include a processing unit or processor or controller, for example, to process signals and/or data generated by imager 46. In another embodiment, the processing unit may be implemented using a separate component within device 40, e.g., controller or processor 47, or may be implemented as an integral part of imager 46, transmitter 41, or another component, or may not be needed. The processing unit may include, for example, a Central Processing Unit (CPU), a Digital Signal Processor (DSP), a microprocessor, a controller, a chip, a microchip, a controller, circuitry, an Integrated Circuit (IC), an Application-Specific Integrated Circuit (ASIC), or any other suitable multi-purpose or specific processor, controller, circuitry or circuit. In one embodiment, for example, the processing unit or controller may be embedded in or integrated with transmitter 41, and may be implemented, for example, using an ASIC.

In some embodiments, device 40 may include one or more illumination sources 42, for example one or more Light Emitting Diodes (LEDs), “white LEDs”, or other suitable light sources. Illumination sources 42 may, for example, illuminate a body lumen or cavity being imaged and/or sensed. An optional optical system 50, including, for example, one or more optical elements, such as one or more lenses or composite lens assemblies, one or more suitable optical filters, or any other suitable optical elements, may optionally be included in device 40 and may aid in focusing reflected light onto imager 46 and/or performing other light processing operations.

Data processor 14 may analyze the data received via external transceiver 12 from device 40, and may be in communication with storage unit 19, e.g., transferring frame data to and from storage unit 19. Data processor 14 may also provide the analyzed data to monitor 18, where a user (e.g., a physician) may view or otherwise use the data. In one embodiment, data processor 14 may be configured for real time processing and/or for post processing to be performed and/or viewed at a later time. In the case that control capability (e.g., delay, timing, etc) is external to device 40, a suitable external device (such as, for example, data processor 14 or external transceiver 12) may transmit one or more control signals to device 40.

Monitor 18 may include, for example, one or more screens, monitors, or suitable display units. Monitor 18, for example, may display one or more images or a stream of images captured and/or transmitted by device 40, e.g., images of the GI tract or of other imaged body lumen or cavity. Additionally or alternatively, monitor 18 may display, for example, control data, location or position data (e.g., data describing or indicating the location or the relative location of device 40), orientation data, and various other suitable data. In one embodiment, for example, both an image and its position (e.g., relative to the body lumen being imaged) or location may be presented using monitor 18 and/or may be stored using storage unit 19. Other systems and methods of storing and/or displaying collected image data and/or other data may be used.

Typically, device 40 may transmit image information in discrete portions. Each portion may typically correspond to an image or a frame; other suitable transmission methods may be used. For example, in some embodiments, device 40 may capture and/or acquire an image once every half second, and may transmit the image data to external transceiver 12. Other constant and/or variable capture rates and/or transmission rates may be used.

Typically, the image data recorded and transmitted may include digital color image data; in alternate embodiments, other image formats (e.g., black and white image data) may be used. In one embodiment, each frame of image data may include 256 rows, each row may include 256 pixels, and each pixel may include data for color and brightness according to known methods. For example, a Bayer color filter may be applied. Other suitable data formats may be used, and other suitable numbers or types of rows, columns, arrays, pixels, sub-pixels, boxes, super-pixels and/or colors may be used.

Optionally, device 40 may include one or more sensors 43, instead of or in addition to a sensor such as imager 46. Sensor 43 may, for example, sense, detect, determine and/or measure one or more values of properties or characteristics of the surrounding of device 40. For example, sensor 43 may include a pH sensor, a temperature sensor, an electrical conductivity sensor, a pressure sensor, or any other known suitable in-vivo sensor.

Although portions of the discussion herein may relate, for exemplary purposes, to pixels, embodiments of the invention are not limited in this regard, and may be used, for example, with relation to multiple pixels, clusters of pixels, image portions, or the like. Furthermore, such pixels or clusters may include, for example, pixels or clusters of an image, pixels or clusters of a set of images, pixels or clusters of an imager, pixels or clusters of a sub-unit of an imager (e.g., a light-sensitive surface of the imager, a CMOS, a CCD, or the like), pixels or clusters represented using analog and/or digital formats, pixels or clusters handled using a post-processing mechanism or software, or the like.

In some embodiments, an image or a set of images acquired by imager 46 may have a relatively Wide Dynamic Range (WDR). For example, the image or set of images may have a first portion which may be relatively saturated, and/or a second portion which may be relatively dark.

In some embodiments, for example, device 40 may handle WDR images by increasing the size of data items transmitted by device 40. For example, a data item transmitted by device 40 may use more than 8 bits (e.g., 9 bits, 10 bits, 11, bits, 12 bits, or the like) to represent a pixel, a cluster of pixels, or an image portion.

In some embodiments, the device 40 may optionally reduce (e.g., slightly reduce) the spatial resolution of acquired images. For example, in one embodiment, device 40 may use an assumption or a rule that a good correlation may exist between a first transmitted data item, which represents a first pixel, and a second transmitted data item, which represents a second, neighboring pixel.

In some embodiments, device 40 may use double-exposure or multiple-exposure system or mechanism for handling WDR images. For example, imager 46 may acquire an image, or the same or substantially the same image, multiple times, e.g., twice or more. In some embodiments, each of the images may be acquired using a different imaging method designed to capture different aspects of a wide dynamic range spectrum; for example high/low light, long/short exposure time, etc. In some embodiments, optionally, a first image may be acquired using a first illumination level, and a second image may be acquired using a second, different, illumination level (e.g., increased illumination, using an increased pulse of light, or the like). In some embodiments, optionally, a first image may be acquired using a first exposure time, and a second image may be acquired using a second, different, exposure time (e.g., increased exposure time). In some embodiments, optionally, two or more images may be acquired with or without changing an image acquisition property (e.g., illumination level, exposure time, or the like), to allow device 40 to acquire twice (or multiple times) the amount of information for an imaged scene or area.

In some embodiments, data may be obtained by device 40 using double-exposure or multiple-exposure, e.g., from a relatively dark region of an image acquired using an increased pulse of light, and/or from a relatively bright or lit region of an image acquired using a decreased (or non-increased) pulse of light. This may, for example, allow device 40 to acquire images having an improved or increased WDR.

In some embodiments, optionally, two images or multiple images acquired using double-exposure or multiple-exposure, respectively, may be stored, arranged or transmitted using interlacing. For example, lines or pixels may be arranged or transmitted alternately, e.g., in two or more interwoven data items. Image interlacing may be performed, for example, by imager 46, processor 47 and/or transmitter 41.

In some embodiments, some of the pixels of imager 46 or some of the pixels of an image acquired by imager 46 (e.g., a first half of the pixels) may have a first responsivity (e.g., “normal” responsivity), and some of the pixels (e.g., a second half of the pixels) may have a second responsivity (e.g., reduced responsivity). This may be achieved, for example, by reducing or otherwise modifying a fill factor (e.g., the percent of area that is exposed to light, or the size of light-sensitive photodiode relative to the surface of the pixel); by increasing or otherwise modifying a well size (e.g., the maximum number of electrons that can be stored in a pixel); by adding or modifying an attenuation layer; or in other suitable methods which may be performed, for example, by imager 46, processor 47 and/or transmitter 41. In some embodiments, this may allow simulation of double-exposure or multiple-exposure of a scene or an imaged area using one image or at one instant, for example, using a slightly-reduced image resolution (e.g., one half resolution at one axis). In some embodiments, a reconstruction process may be performed (e.g., by workstation 17 or processor 14), to overcome or compensate a possible image degradation, e.g., thereby allowing imager 46 to acquire WDR images without necessarily increasing (e.g., doubling) the amount of data transmitted by the device 40.

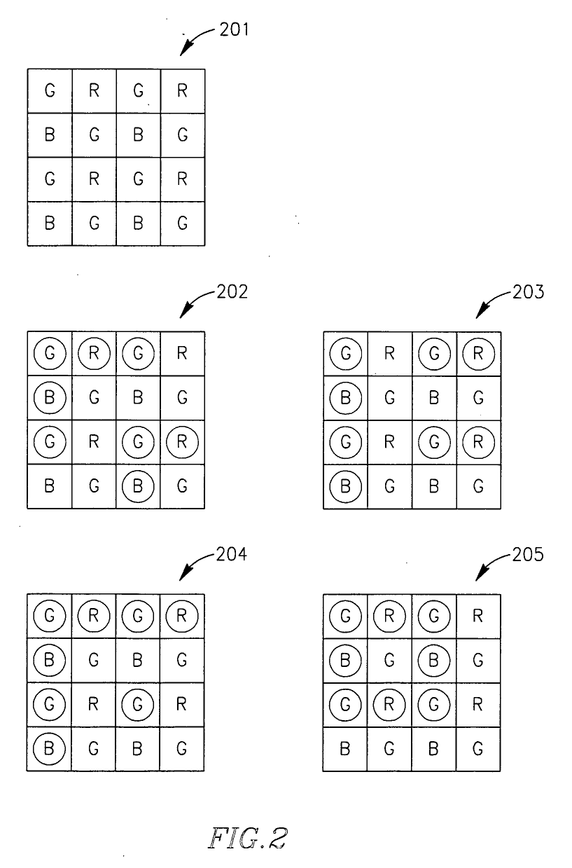

Reference is made to FIG. 2, which schematically illustrates pixel groupings 201-205 in accordance with some embodiments of the invention. The groupings 201-205 may be used, for example, for grouping of pixels or clusters of an image or an imager (e.g., imager 46).

In some embodiments, different groups of pixels may have different sensitivity or other characteristics, such that, for example, each group may capture, or may be more sensitive or less sensitive, in a different area or portion of the WDR. For example, some pixels may be highly sensitive to light, and others less sensitive to light. In some embodiments, pixels or clusters (or data representing pixels or clusters) may be grouped into, for example, two or more groups, e.g., in accordance with grouping rules, grouping constraints, a pre-defined pattern (e.g., Bayer pattern), or the like. For example, in one embodiment, pixels may be arranged in accordance with Bayer pattern, such that half of the total number of pixels are green (G), a quarter of the total number of pixels are red (R), and a quarter of the total number of pixels are blue (B). Accordingly, as shown in arrangement 201, a first line of pixels may read GRGR, a second line of pixels may read BGBG, etc.

In some embodiments, a grouping rule may be defined and used such that a pre-defined resolution (or ratio) of all bands is maintained (e.g., over an entire image or imager) in all groups. For example, as shown in arrangements 202-205, circled pixels may belong to a first group, and non-circled pixels may belong to a second group. In some embodiments, the number of green pixels in the first group may be equal to the number of green pixels in the second group; the number of red pixels in the first group may be equal to the number of red pixels in the second group; and the number of blue pixels in the first group may be equal to the number of blue pixels in the second group. Other suitable constraints, rules or grouping rules may be used, and other sizes or types of arrangements, pixel clusters, repetition blocks or matrices may be used in accordance with embodiments of the invention.

In some embodiments, pixels of the first group may be low-responsivity pixels or reduced-responsivity pixels (hereinafter, “low-responsivity pixels”), whereas pixels of the second group may be “normal”-responsivity pixels or increased-responsivity pixels (hereinafter, “normal-responsivity pixels”), or vice versa. Other properties or characteristics may be assigned to, or associated with, one or more groups of pixels.

In some embodiments, more than two groups of pixels with different responsiveness or sensitivity may be used. Different responsiveness or sensitivity may be achieved by the design of individual pixels in an imager, by circuitry, or by post-processing software.

In some embodiments, image information may be reconstructed by processor 14 based on data received, for example, by receiver/recorder 12 from device 40. Different groups of image data (e.g., obtained from different pixel groups, different images, or the like), having or having captured different portions of a WDR spectrum, may be recombined, reconstructed, merged, or otherwise handled, for example, to create or yield a WDR image. In one embodiment, for example, if normal-responsivity pixels at an inspected region are not saturated, then the inspected region or a larger portion of the image (e.g., substantially the entire image) may be reconstructed based on the normal-responsivity pixels, optionally taking into account edge indications or edge clues which may be present in the low-responsivity pixels. In another embodiment, for example, if the normal-responsivity pixels are saturated, then only low-responsivity pixels may be used for reconstruction. Various suitable reconstruction algorithms may be used in accordance with embodiments of the invention, for example, taking into account a grouping or a grouping pattern (e.g., a “dilution” pattern) which may be used.

In some embodiments, imager 46 may handle scenes, images or frames in which data of a first portion (e.g., a first half) includes relatively high values (e.g., close to saturation) and data of a second portion (e.g., a second half) represents a relatively dark area.

In some embodiments, an Automatic Light Control (ALC) unit 91 may optionally be included in device 40 (e.g., as part of imager 46 or as a sub-unit of device 40). ALC 91 may, for example, determine exposure time and/or gain, e.g., to avoid or decrease possible saturation. Gain calculation may be performed, for example, to allow an improved or optimal use of an Analog to Digital (A/D) converter 92, which may be included in device 40 (e.g., as part of imager 46 or as a sub-unit of device 40). For example, in one embodiment, gain calculation may be performed in device 40 prior to A/D conversion.

In some embodiments, ALC 91 or other components of device 40 may be similar to embodiments described in U.S. patent application Ser. No. 10/202,608, entitled “Apparatus and Method for Controlling Illumination in an In-Vivo Imaging Device”, filed on Jul. 25, 2002, published on Jun. 26, 2003 as U.S. Patent Application Publication Number 2003/0117491, which is assigned to the common assignee of the present invention and which is hereby fully incorporated by reference.

In one embodiment, ALC 91 may determine gain globally, e.g., with regard to substantially an entire image, scene or frame. In another embodiment, ALC 91 may determine gain locally, e.g., with regard to a portion of an image, a pixel, multiple pixels, a cluster of pixels, or other areas or sub-areas of an image.

In some embodiments, gain calculation and determination may be performed by units other than ALC 91, for example, by imager 46, transmitter 41, or processor 47. In some embodiments, A/D conversion may be performed by units other than A/D converter 92, for example, by imager 46, transmitter 41, or processor 47

In some embodiments, device 40 may determine and use a relatively higher gain value in a dark (or relatively darker) portion of an image, thereby reducing possible quantization noise. In one embodiment, for example, a value (e.g., analog pixel value) calculated or determined with regard to a first pixel, may be used for determining or calculating gain with regard to a second pixel, e.g., a neighboring or consecutive pixel. For example, in some embodiments, if the value of a first pixel is low or relatively low, e.g. below a certain and/or pre-determined threshold, then the gain (e.g., analog gain) of a second (e.g., neighboring or consecutive) pixel may be increased. In some embodiments, if the value (e.g., analog value) of a first pixel is high or relatively high, e.g. above a certain and/or pre-determined threshold, then the gain of a second (e.g., neighboring or consecutive) pixel may be reduced. Other determinations or rules may be used for local gain calculations. In some embodiments, this may allow, for example, improved or increased Signal to Noise Ration (SNR), and/or avoiding or reducing possible saturation.

In some embodiments, for example, Gain_{Old} may represent the gain of a first pixel, and Gain_{New} may represent the gain of a second (e.g., neighboring or consecutive) pixel. The first pixel may have a value of Value_{Old}. Gain_{Max} may represent a maximum gain level (e.g., 8 or 16 or other suitable values). TH1 may represent a first threshold value, and TH2 may represent a second threshold value; in one embodiment, for example, TH1 may be smaller than TH2.

In some embodiments, Gain_{New} may be determined or calculated based on, for example, Gain_{Old}, Value_{Old}, Gain_{Max}, TH1, TH2, and/or other suitable parameters. For example, in one embodiment, the following calculation may be used: if Value_{Old} is smaller than TH1, then Gain_{New} may be equal to the smaller of Gain_{Max} and twice the value of Gain_{Old}; otherwise, if Value_{Old} is greater than TH2, then Gain_{New} may be equal to the greater of one or one half of Gain_{Old}; otherwise, Gain_{New} may be equal to Gain_{Old}. Other suitable rules, conditions or formula may be used in accordance with embodiments of the invention.

In some embodiments, for example, the gain (e.g., Gain_{New}) may not be smaller than one. In some embodiments, for example, TH1 and TH2 may be pre-defined in accordance with specific implementations; for example, in one embodiment, TH1 may be equal to 96 and TH2 may be equal to 224. In some embodiments, for example, TH1 may be smaller than 128. In some embodiments, for example, TH2 may be close or relatively close to 255. In some embodiments, for example, the further TH2 is from 255, the greater the possibility of avoiding saturation or unnecessary (e.g., false) saturation. Other suitable values or ranges of values may be used.

In some embodiments, if a determined gain of a first pixel (e.g., Gain_{Old}) results in saturation, then the gain of a second (e.g., neighboring or consecutive) pixel (e.g., Gain_{New}) may be calculated such as to avoid or reduce saturation, for example, in accordance with the conditions discussed herein and/or other suitable conditions or rules.

In some embodiment, calculation and determination of local gain (e.g., per pixel, per multiple pixels, per cluster of pixels, or the like) may be performed, for example, by a Local Gain Control (LGC) unit 93 which may optionally be included in device 40 (e.g., as part of imager 46 or as a sub-unit of device 40). In some embodiments, calculation and determination of local gain may be performed by units other than LGC 92, for example, by imager 46, transmitter 41, or processor 47.

In some embodiments, local gain may be calculated or determined separately with regard to various or separate color channels. In some embodiments, for substantially every line of data, the initial gain for the first pixel may be defined or pre-defined (e.g., such that, for example, the first pixel in every line may have a gain of “2”), since data acquired from the previous line may not be used to determine the gain for the subsequent line.

In some embodiments, pixel values may be reconstructed (e.g., by workstation 17 or processor 14), for example, based on TH1 and TH2. In one embodiment, for example, values of TH1 and TH2 may be transmitted by device 40, or may be pre-defined in device 40 and/or workstation 17. In one embodiment, optionally, a first pixel may have a pre-defined gain (e.g., equal to 1 or other pre-defined value), to allow or facilitate gain calculation with regard to other (e.g., consecutive or neighboring) pixels.

Reference is made to tables 1-3, which are three tables of exemplary image data which may be avoided or cured by some embodiments of the invention.

TABLE 1

| | | | | | | |
|---|---|---|---|---|---|---|
| Original data | 115.4 | 115.4 | 115.4 | 115.4 | 115.4 | 115.4 |
| Actual value | 115 | 231 | 115 | 231 | 115 | 231 |
| Actual gain | 1 | 2 | 1 | 2 | 1 | 2 |
| Estimated data | 115 | 115.5 | 115 | 115.5 | 115 | 115.5 |

TABLE 2

| | | | | | | |
|---|---|---|---|---|---|---|
| Original data | 80 | 80 | 80 | 130 | 130 | 130 |
| Actual value | 80 | 160 | 160 | 255 | 130 | 130 |
| Actual gain | 1 | 2 | 2 | 2 | 1 | 1 |
| Estimated data | 80 | 80 | 80 | 128 | 130 | 130 |

TABLE 3

| | | | | | | |
|---|---|---|---|---|---|---|
| Original data | 112 | 112.5 | 113 | 113.5 | 114 | 114.5 |
| Actual value | 224 | 112 | 113 | 113 | 114 | 114 |
| Actual gain | 2 | 1 | 1 | 1 | 1 | 1 |
| Estimated data | 112 | 112 | 113 | 113 | 114 | 114 |
| Actual value without LGC | 224 | 225 | 226 | 227 | 228 | 229 |
| Actual gain without LGC | 2 | 2 | 2 | 2 | 2 | 2 |
| Estimated value without LGC | 112 | 112.5 | 113 | 113.5 | 114 | 114.5 |

In Tables 1-3, “original data” may indicate the data as actually sensed (e.g., imaged) by the imager 46, for example, the analog data sensed; “actual value” may indicate the data as transmitted by device 40, for example, the digital data after the Analog to Digital (A/D) conversion and after a set gain; “actual gain” may indicate the gain associated with the data; “estimated data” may include the data as estimated or reconstructed (e.g., by workstation 17 or processor 14); and “without LGC” may indicate that transmitted data is not subject to Local Gain Control (LGC) and may have a constant pre-determined gain value (e.g., equal to the gain of the first pixel in the row).

In some embodiments, the values of TH1 and TH2 may be determined or selected, and LGC may be used, such as to avoid or reduce an “unstable” data structure. As shown in Table 1, an unstable data structure may include, for example, a sequence of estimated or reconstructed data in which two values (e.g., 115 and 115.5) alternate along a series of consecutive pixels although the originally imaged data included a repeating or substantially constant value (e.g., 115.4). For example, in one embodiment, if TH1 is equal to 120 -and TH2 is equal to 224, then an unstable data structure may be reconstructed or estimated, as shown in Table 1. Therefore, in some embodiments, TH1 and TH2 may be set to other values, or another compensating or correcting mechanism may be used, to avoid or cure an unstable data structure.

In some embodiments, LGC may be used to avoid or reduce a “false” saturation data structure. As shown in Table 2, a false saturation data structure may include, for example, using a gain value that results in saturation of the estimated data, although the original data need not result in saturation. For example, as indicated at the fourth column from the right, if the original data is equal to 80, then it may be correct that device 40 transmits an actual value of 160 and a gain of 2. However, as indicated at the third column from the right, if the original data is equal to 130, then it may be incorrect that device 40 transmits an actual value of 255 and a gain of 2, thereby resulting in a false saturation and estimated data equal to 128 (and not 130, which is the original data at the first row of Table 2 on the third column from the right). Therefore, in some embodiments, the LGC mechanism or its parameters may be fine-tuned, or another compensating or correcting mechanism may be used, to avoid or cure a false saturation data structure.

In some embodiments, LGC may be used to avoid or reduce an over-quantization of data. As shown in Table 3, if the original data includes, for example, gradually increasing values, then using a LGC mechanism may result in over-quantization of the estimated data. Therefore, in some embodiments, the LGC mechanism or its parameters may be fine-tuned, or another compensating or correcting mechanism may be used, or the device 40 may avoid using a LGC mechanism, to avoid or cure over-quantization of data.

In some embodiments, other suitable compensating mechanisms may be used. For example, in one embodiment, less quantization noise may be achieved at image areas having low intensity. In some embodiments, for example, transition from dark or very dark regions to bright or very bright regions (or vice versa) may cause false saturation. A pre-processing mechanism (e.g., detecting a “255” value and determining a gain equal to 1) and/or a post-processing mechanism (e.g., by workstation 17 or processor 14) may be used to avoid such situations. In one embodiment, such post-processing mechanism may be configured, for example, to handle “255” values in accordance with a pre-defined algorithm.

Table 4 includes exemplary image data in accordance with some embodiments of the invention, allowing, for example, relatively more accurate data and avoiding a potential false saturation level for the pixel having an original value of “200”.

TABLE 4

| | | | | | | |
|---|---|---|---|---|---|---|
| Original data | 80.5 | 81 | 123 | 200 | 90 | 95.5 |
| Actual value | 161 | 162 | 226 | 200 | 90 | 191 |
| Actual gain | 2 | 2 | 2 | 1 | 1 | 2 |
| Estimated data | 80.5 | 81 | 123 | 200 | 90 | 95.5 |
| Estimated data using | 80 | 81 | 123 | 110 | 90 | 95 |
| a constant gain of “1” | | | | | | |

According to some embodiments, threshold levels may be such that, for example, the gain can be increased to 4, 8, 16, or other values.

In some embodiments, WDR images acquired by device 40 may originally be represented by data having, for example, 8 bits per pixel. However, after the sensed data is handled by device 40 (e.g., using double-exposure and/or LGC), representation of the data may require a larger number of bits (e.g., 10 bits, 11 bits, 12 bits, or the like). For example, in one embodiment, device 40 may use 8 bits to represent a value of a pixel (e.g., in the range of 0 to 255), and additional bits to represent the gain of the pixel.

In one embodiment, for example, three bits may be used to represent possible gain values of 1, 2, 4, 8 and 16. For example, the three bits “000” may represent a gain of 1; the three bits “001” may represent a gain of 2; the three bits “010” may represent a gain of 4; the three bits “011” may represent a gain of 8; and the three bits “100” may represent a gain of 16. Other suitable representations may be used; for example, two bits may be used to represent possible values of 1, 2 and 4.

In some embodiments, device 40 may compress the data (e.g., using processor 47) prior to transmitting it (e.g., using transmitter 41). In one embodiment, the compression algorithm may require an 8-bit data structure, may operate efficiently or relatively efficiently on 8-bit data structures, and may not operate efficiently or relatively efficiently on data structures having other sizes (e.g., 10 bits, 11 bits, 12 bits, or the like) (“oversized data item”). Therefore, in some embodiments, device 40 may further handle or modify oversized data items prior to their compression and transmission, for example, to allow the data to be more compatible with a pre-defined compression algorithm possibly used by device 40.

In one embodiment, device 40 may apply the compression algorithm on “wrapped” data items, such that additional bits of data (e.g., beyond the original 8 bits) of an oversized data item are considered part of the next data item, and/or such that oversized data items may be “broken” or split over several 8-bit sequences. In some embodiments, such handling of oversized data item may not allow gain data to be apparent or readily available for workstation 17.

In another embodiment, oversized data items may be handled by, for example, transforming the in-vivo system to an increased bit-space (e.g., 10 bits space, 11 bits space, 12 bits space, or the like). In one embodiment, this may result in a possible decrease in compression efficiency; in another embodiment, other compensating mechanisms may be used, or compression need not be used such that oversized data items may be transmitted uncompressed.

In yet another embodiment, oversized data items may be represented using floating-point representation, or another representation scheme which may be similar to floating-point representation, for example, having a mantissa field and an exponent field. In one embodiment, for example, oversized data items may be converted (e.g., by processor 47 or imager 46) to floating-point representation, and may then be compressed and transmitted. In some embodiments, for example, a certain number of bits (e.g., two bits or three bits) of the floating-point representation may be used to indicate the gain, and the rest of the bits (e.g., six bits or five bits) may be used to indicate the pixel value. In some embodiments, optionally, one or two last bits (e.g., least significant bits) of the original data may be discarded in order to achieve floating-point representation. In some embodiments, for example, a floating-point type representation of an oversized data item may include an exponent component corresponding to a gain value and a mantissa component corresponding to a pixel value.

In some embodiments, a floating-point type representation may be used such that an 8-bit data item may include three most-significant bits (e.g., representing an exponent field) and five least-significant bits (e.g., representing a mantissa). Other number of bits may be used. In one embodiment, for example, the exponent field may be used to indicate the position of the first occurrence of “1” in the oversized data item, and the mantissa field may be used to indicate the next five bits (e.g., starting with the first occurrence of “1”) in the oversized data item. Other suitable compensating or representation methods may be used.

In some embodiments, the representation of oversized data items may be, for example, monotonic and/or unique. For example, if a certain analog input is sampled using two different digital gain values, then the digital output representation may be substantially the same, not taking into account possible quantization noise. In one embodiment, if two different analog inputs are sampled (e.g., Value1 and Value2), then their digital floating-point representations (e.g., FP1 and FP2, respectively) may maintain their relational size, for example, such that if Value1 is greater than Value2, then FP1 is greater than FP2, and vice versa.

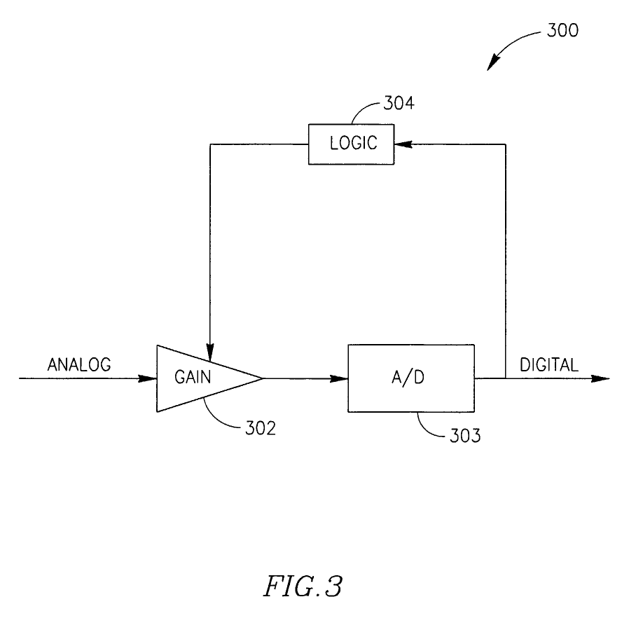

Reference is made to FIG. 3, which schematically illustrates a block diagram of a circuit 300 in accordance with some embodiments of the invention. Circuit 300 may be, for example, part of imager 46 of FIG. 1, or part or sub-unit of device 40 of FIG. 1.

Circuit 300 may receive analog input, for example, sensed image data in analog format. The analog input may be transferred to a gain stage 302, prior to performing A/D conversion by an A/D converter 303. Digital output of the A/D converter 303 with regard to a first pixel, may be used by a logic unit 304 and/or gain stage 302 to determine local gain for a second (e.g., consecutive or neighboring) pixel. In one embodiment, the gain of a first pixel in a line may be pre-defined or preset (e.g., to a value of “1” or “2”); and the gain of a consecutive pixel (e.g., in the same line) may be determined based on the value of the previous pixel. In one embodiment, local gain determination may include serial scanning of consecutive pixels in a line, or other suitable operations to determine gain of a first pixel based on a value of a second pixel.

Circuit 300 may include other suitable components, and may be implemented, for example, as part of imager 46, processor 47, transmitter 41 and/or device 40.

Reference is made to Tables 5A-5D which are four exemplary tables of floating-point representations of oversized data items in accordance with some embodiments of the invention.

TABLE 5A

| Floating Point | Actual Range of | |
|---|---|---|
| Representation | Values | Resolution |
| 0XXXXXXX | 0-127 | 1 |
| 100XXXXX | 128-190 | 2 |
| 101XXXXX | 192-316 | 4 |
| 110XXXXX | 320-568 | 8 |
| 111XXXXX | 576-1072 | 16 |

TABLE 5B

| Floating Point | Actual Range of | |
|---|---|---|
| Representation | Values | Resolution |
| 00XXXXXX | 0-63 | 1 |
| 01XXXXXX | 64-190 | 2 |
| 100XXXXX | 192-316 | 4 |
| 101XXXXX | 320-568 | 8 |
| 110XXXXX | 576-1072 | 16 |
| 111XXXXX | 1088-2080 | 32 |

TABLE 5C

| Floating Point | Actual Range of | |
|---|---|---|
| Representation | Values | Resolution |
| 00XXXXXX | 0-63 | 1 |
| 010XXXXX | 64-126 | 2 |
| 011XXXXX | 128-252 | 4 |
| 100XXXXX | 256-504 | 8 |
| 101XXXXX | 512-1008 | 16 |
| 110XXXXX | 1024-2016 | 32 |
| 111XXXXX | 2048-4032 | 64 |

TABLE 5D

| Floating Point | Actual Range of | |
|---|---|---|
| Representation | Values | Resolution |
| 000XXXXX | 0-31 | 1 |
| 001XXXXX | 32-94 | 2 |
| 010XXXXX | 96-220 | 4 |
| 011XXXXX | 224-472 | 8 |
| 100XXXXX | 480-976 | 16 |
| 101XXXXX | 992-1984 | 32 |
| 110XXXXX | 2016-4000 | 64 |
| 111XXXXX | 4064-8032 | 128 |

Tables 5A and 5B may be used, for example, in conjunction with oversized data items having 10 or 11 bits; Tables 5C and 5D may be used, for example, in conjunction with oversized data items having 12 or 13 bits. Other tables may be used, to accommodate oversized data items having other number of bits.

In Tables 5A-5D, the left column indicates the floating-point representation, such that the left-most characters (e.g., having values of “0” or “1”) indicate a gain code or gain value, whereas the right-most characters (e.g., shown as “X” characters) indicate bits (e.g., the most-significant bits) of the pixel value. The center column indicates the corresponding actual ranges of values which may be represented, and the right column indicates the corresponding resolution. Other suitable values, ranges, representations, resolutions and/or tables may be used.

Tables 6A-6E are five exemplary tables of floating-point representations of oversized data items in accordance with some embodiments of the invention.

TABLE 6A

| Fixed point | Floating point | |
|---|---|---|
| representation | representation | Remarks |
| 001A₈A₇A₆A₅A₄A₃A₂00 | 11A₈A₇A₆A₅A₄A₃ | X1, loosing last bit |
| 0001A₇A₆A₅A₄A₃A₂A₁0 | 10A₇A₆A₅A₄A₃A₂ | X2, loosing last bit |
| 0000A₇A₆A₅A₄A₃A₂A₁A₀ | 0A₇A₆A₅A₄A₃A₂A₁ | X4, loosing last bit |

TABLE 6B

| Fixed point | Floating point | |
|---|---|---|
| representation | representation | Remarks |
| 001A₈A₇A₆A₅A₄A₃A₂00 | 111A₈A₇A₆A₅A₄ | X1, loosing last two bits |
| 0001A₇A₆A₅A₄A₃A₂A₁0 | 110A₇A₆A₅A₄A₃ | X2, loosing last two bits |
| 00001A₆A₅A₄A₃A₂A₁A₀ | 10A₆A₅A₄A₃A₂A₁ | X4, loosing last bit |
| 00000A₆A₅A₄A₃A₂A₁A₀ | 0A₆A₅A₄A₃A₂A₁A₀ | X4, no lose |

TABLE 6C

| Fixed point | Floating point | |
|---|---|---|
| representation | representation | Remarks |
| 01A₉A₈A₇A₆A₅A₄A₃000 | 111A₉A₈A₇A₆A₅ | X1, loosing last two bits |
| 001A₈A₇A₆A₅A₄A₃A₂00 | 110A₈A₇A₆A₅A₄ | X2, loosing last two bits |
| 0001A₇A₆A₅A₄A₃A₂A₁0 | 101A₇A₆A₅A₄A₃ | X4, loosing last two bits |
| 00001A₆A₅A₄A₃A₂A₁A₀ | 100A₆A₅A₄A₃A₂ | X8, loosing last two bits |
| 00000A₆A₅A₄A₃A₂A₁A₀ | 0A₆A₅A₄A₃A₂A₁A₀ | X8, no lose |

TABLE 6D

| Fixed point | Floating point | |
|---|---|---|
| representation | representation | Remarks |
| 01A₉A₈A₇A₆A₅A₄A₃000 | 111A₉A₈A₇A₆A₅ | X1, loosing last two bits |
| 001A₈A₇A₆A₅A₄A₃A₂00 | 110A₈A₇A₆A₅A₄ | X2, loosing last two bits |
| 0001A₇A₆A₅A₄A₃A₂A₁0 | 101A₇A₆A₅A₄A₃ | X4, loosing last two bits |
| 000011A₅A₄A₃A₂A₁A₀ | 100A₅A₄A₃A₂A₁ | X8, loosing last bit |
| 000010A₅A₄A₃A₂A₁A₀ | 011A₅A₄A₃A₂A₁ | X8, loosing last bit |
| 000001A₅A₄A₃A₂A₁A₀ | 010A₅A₄A₃A₂A₁ | X8, loosing last bit |
| 000000A₅A₄A₃A₂A₁A₀ | 00A₅A₄A₃A₂A₁A₀ | X8, no lose |

TABLE 6E

| Fixed point | Floating point | |
|---|---|---|
| representation | representation | Remarks |
| 01A₉A₈A₇A₆A₅A₄A₃000 | 111A₉A₈A₇A₆A₅ | X1, loosing last two bits |
| 001A₈A₇A₆A₅A₄A₃A₂00 | 110A₈A₇A₆A₅A₄ | X2, loosing last two bits |
| 0001A₇A₆A₅A₄A₃A₂A₁0 | 10A₇A₆A₅A₄A₃A₂ | X4, loosing last bit |
| 0000A₇A₆A₅A₄A₃A₂A₁A₀ | 0A₇A₆A₅A₄A₃A₂A₁ | X8, loosing last bit |

Tables 6A and 6B may be used, for example, in conjunction with oversized data items having 10 bits; Tables 6C-6E may be used, for example, in conjunction with oversized data items having 11 bits. Other tables may be used, to accommodate oversized data items having other number of bits.

In Tables 6A-6E, the left column indicates fixed-point representation of oversized data items. The center column indicates the floating-point representation, such that the left-most characters (e.g., having values of “0” or “1”) indicate a gain code or gain value, whereas the right-most characters (e.g., shown as “A” characters) indicate bits (e.g., the most-significant bits) of the pixel value. The right column indicates how many bits (e.g., least-significant bits) of the pixel value may be discarded, and the gain level (e.g., “X1” indicating a gain of 1, “X2” indicates a gain of 2, etc.). Other suitable values, representations, ranges, resolutions and/or tables may be used.

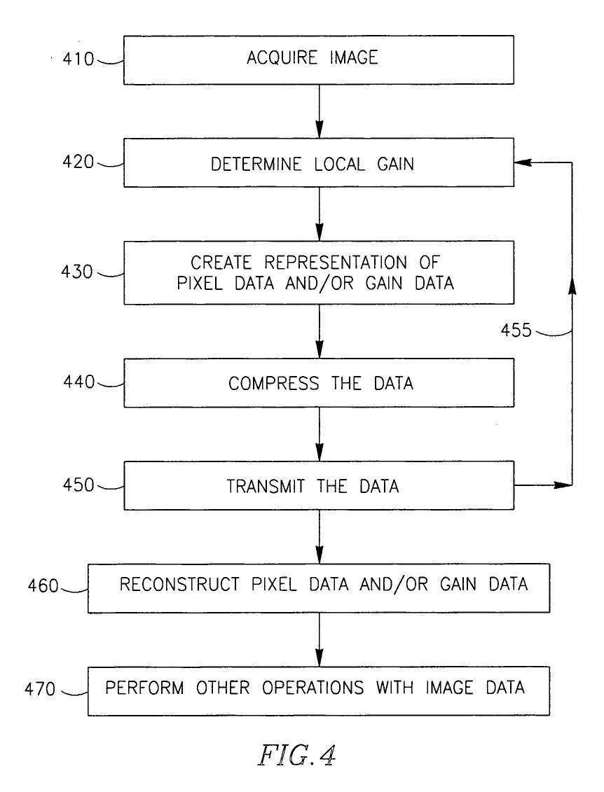

FIG. 4 is a flow-chart diagram of a method of imaging in accordance with some embodiments of the invention. The method may be used, for example, in association with the system of FIG. 1, with device 40 of FIG. 1, with one or more in-vivo imaging devices (which may be, but need not be, similar to device 40), with imager 46 of FIG. 1, and/or with other suitable imagers, devices and/or systems for in-vivo imaging or in-vivo sensing. A method according to embodiments of the invention need not be used in an in-vivo context.

In some embodiments, as indicated at box 410, the method may optionally include, for example, acquiring in-vivo an image or multiple images. This may include, for example, acquiring in-vivo one or more WDR images, e.g., using double-exposure or multiple-exposure.

In some embodiments, as indicated at box 420, the method may optionally include, for example, determining local gain. This may include, for example, determining gain with regard to a portion of an image, a pixel, multiple pixels, a cluster of pixels, or other areas or sub-areas of an image. In some embodiments, for example, gain of a first pixel may optionally be used for determining gain of a second (e.g., neighboring or consecutive) pixel. In some embodiments, for example, local gain calculation may use one or more compensating mechanisms, for example, to avoid or reduce “false” saturation, to avoid or reduce an “unstable” data structure, to avoid or reduce over-quantization of data, or the like.

In some embodiments, as indicated at box 430, the method may optionally include, for example, creating a representation of pixel data and/or gain data (e.g., local gain data). This may include, for example, creating oversize data items, mapping or reformatting oversize data items in accordance with a mapping or reformatting table, encoding oversize data items in accordance with an encoding table, modifying or transferring fixed-point data items to floating-point data items, or the like.

In some embodiments, as indicated at box 440, the method may optionally include, for example, compressing the data, e.g., pixel data, gain data, data items having pixel data and gain data, or the like.

In some embodiments, as indicated at box 450, the method may optionally include, for example, transmitting the data, e.g., from an in-vivo imaging device to an external receiver/recorder.

In some embodiments, as indicated by an arrow 455, the method may optionally include, for example, repeating one or more of the above operations, e.g., the operations of boxes 920, 930, 940 and/or 950. This may optionally allow, for example, serial scanning of images, pixels, or image portions.

In some embodiments, as indicated at box 460, the method may optionally include, for example, reconstructing pixel data and/or gain data (e.g., local gain data), for example, by an external processor or workstation. In some embodiments, gain of a first pixel may be determined or calculated based on gain and/or value of a second (e.g., neighboring or consecutive) pixel. In other embodiments of the invention, reconstruction of gain data (e.g., local gain data) may optionally be performed prior to compression.

In some embodiments, as indicated at box 470, the method may optionally include, for example, performing other operations with image data (e.g., pixel data and/or gain data). This may include, for example, displaying image data on a monitor, storing image data in a storage unit, processing or analyzing image data by a processor, or the like.

It is noted that some or all of the above-mentioned operations may be performed substantially in real time, e.g., during the operation of the in-vivo imaging device, during the time in which the in-vivo imaging device operates and/or captures images and/or transmits images, typically without interruption to the operation of the in-vivo imaging device.

Other suitable operations or sets of operations may be used in accordance with embodiments of the invention.

A device, system and method in accordance with some embodiments of the invention may be used, for example, in conjunction with a device which may be inserted into a human body. However, the scope of the present invention is not limited in this regard. For example, some embodiments of the invention may be used in conjunction with a device which may be inserted into a non-human body or an animal body.

While certain features of the invention have been illustrated and described herein, many modifications, substitutions, changes, and equivalents may occur to those of ordinary skill in the art. It is, therefore, to be understood that the appended claims are intended to cover all such modifications and changes as fall within the true spirit of the invention.

1-12. (canceled)

13. An in-vivo imaging system comprising a local gain control unit to determine local gain for a portion of an image captured by an imager.

14. The in-vivo imaging system of claim 13, wherein said portion of an image comprises a pixel.

15. The in-vivo imaging system of claim 13, wherein the local gain control unit is to determine gain of a first pixel based on gain of a second pixel.

16. The in-vivo imaging system of claim 13, wherein the local gain control unit is to determine local gain of a pixel based on a comparison of a value of said pixel with a threshold value.

17. The in-vivo imaging system of claim 14, comprising an in-vivo device comprising a processor to create a representation of said local gain and at least a portion of a value of said pixel.

18. The in-vivo imaging system of claim 17, wherein said representation is a floating-point type representation.

19. The in-vivo imaging system of claim 18, wherein the processor is to compress the representation.

20. The in-vivo imaging system of claim 19, comprising a transmitter to transmit the compressed representation.

21. (canceled)

22. (canceled)

23. (canceled)

24. The in-vivo imaging system of claim 14, comprising:
a receiver to receive from an in-vivo imaging device a representation of said local gain of a pixel and at least a portion of a value of said pixel.

25. The in-vivo imaging system of claim 19, comprising:
a data processor to reconstruct said value of said pixel and said gain of said pixel based on said representation.

26. (canceled)

27. (canceled)

28. (canceled)

29. A method for wide dynamic range imaging with an in-vivo imaging device comprising:
capturing in-vivo a first and second portion of an image, wherein the first portion is captured at a first pixel gain and the second portion is captured at a second pixel gain.

30. The method of claim 29, wherein the first and second portions correspond to first and second aspects of a wide dynamic range image, respectively.

31. The method of claim 29 comprising capturing the image with an imager including a first group of low-responsivity pixels and a second group of increased-responsivity pixels.

32. The method of claim 31 comprising capturing the first portion of the image with the low-responsivity pixels and the second portion of the image with the increased-responsivity pixels.

33. The method of claim 29, wherein the first portion of the image is captured with a plurality of sets of color pixels.

34. The method of claim 29, wherein the first and second portion of the image include an equal number of pixels with a pre-defined color.

35. The method of claim 29 comprising representing a pixel of the wide dynamic range image using more than eight bits.

36. The method of claim 35 comprising compressing the representation of the pixel comprising more than eight bits.

37. The method of claim 29, comprising capturing the image with a swallowable in-vivo imaging device.
